(19) United States
(12) Patent Application Publication
(10) Pub. No.: US 2007/0167708 A1
(43) Pub. Date: Jul. 19, 2007

(54) Marker for a navigation system and method for detecting a marker

(75) Inventors: Andreas BLUMHOFER, Neubiberg (DE); Johannes MANUS, Munchen (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(73) Assignee: BRAINLAB AG, Feldkirchen (DE)

(21) Appl. No.: 11/548,848

(22) Filed: Oct. 12, 2006

Related U.S. Application Data
(60) Provisional application No. 60/735,026, filed on Nov. 9, 2005.

(30) Foreign Application Priority Data
Oct. 12, 2005 (EP) 05022214.0
Oct. 12, 2005 (EP) 04022214.0

(51) Int. Cl. A61B 5/05

(52) U.S. Cl. 600/407

(57) ABSTRACT

A marker for a navigation system includes a reflective surface, and a light-absorbing, non-reflective or low-reflective object arranged at a defined distance from the reflective surface.

### RELATED APPLICATION DATA

This application claims priority of U.S. Provisional Application No. 60/735,026 filed on Nov. 9, 2005, which is incorporated herein by reference in its entirety.

### FIELD OF THE INVENTION

The present invention relates to a marker and, more particularly, to a marker array that is trackable by a medical navigation system, and a method for detecting the position of a marker.

### BACKGROUND OF THE INVENTION

Markers, for example, are used in image-assisted surgical methods, e.g., image-guided surgery (IGS), to ascertain a position of surgical instruments or bodies, wherein a number of markers in the form of a reference star are connected to the surgical instruments and/or bodies. The position may be determined by optically detecting light reflected by the markers. From the ascertained position, image guided surgery can be performed.

Spherically shaped passive markers typically do not exhibit homogenous reflectivity, since a retro-reflective film or coating applied to the surface of the sphere only exhibits good reflectivity within a particular angular range. However, a critical angle for reflectivity cannot be accurately reproduced such that the reflectivity at the visible periphery of the marker can be definitely determined. The heterogeneity of the reflectivity of a marker is additionally compounded by applying the film to a spherical base of the marker, which can lead to undefined distortions in the reflective film. The accuracy of a tracking system in determining a position of the marker is consequently limited, in particular when marker recognition algorithms are used that are based on detecting a barycenter.

If flat markers are used, then, when the barycenter of reflection of a circular reflective marker lying obliquely with respect to a detection plane is detected, positional inaccuracy can be achieved from the circular reflective marker projected onto the camera plane as an ellipse, the barycenter of which does not lie exactly at the center point of the circular marker. Inaccuracies can also occur when applying the film.

A spherical retro-reflective video marker is known from GB-2,404,453 A which can be fixed to an object for video tracking.

### SUMMARY OF THE INVENTION

A marker or marker array for use in conjunction with a navigation system, for example, exhibits a light-reflective surface formed as a plane or flat surface that can include one or more curves, wherein a light-absorbing, non-transparent or opaque object is attached at a defined distance (e.g., 0, 0.5, 1.0 or more than 1.0 mm) from the reflective surface. Alternatively, the object location may be variably positioned relative to the reflective surface. The light-absorbing or non-reflective object can be any known three-dimensional object such as, for example, a sphere, a cuboid, an ellipsoid or other object, the shape of which can be simply ascertained and mathematically described. Preferably, the object is fixedly connected to the reflective surface, e.g., by means of a spacer or rod which can be arranged at a defined point on the reflective surface, such as for example in the center of the reflective surface, and can be directed to the center point of a sphere serving as the absorbing object.

The marker array described herein enables greater accuracy in position recognition. For example, when using a sphere as the light-absorbing object, the marker image, which can be detected by a camera, is an exact circle with a sharp and easily recognizable outline and, therefore, more reliable circle detection algorithms can be used. The marker image also has a high contrast, e.g., a sharp contour at the transition between a reflective area recognized by a camera and the reflective area shielded by the absorbing object, which can be recognized as a dark area, for example. A reflective area also can be used with a body which has a different and, for example, poorer reflectivity than a reflective area arranged on the body, wherein the different luminosities or light intensities of the detected two-dimensional object and reflection area images can be recognized and evaluated by a navigation system.

By comparison, a strong contrast cannot be realized with known markers, in particular with film-covered or coated markers.

When using the marker, the marker image, which can be detected by a camera, is larger for the same size marker sphere, since the reflective area lying in the field of vision of the detection camera is completely covered by the sphere, whereas a marker sphere coated with a reflective substance no longer generates a full reflection in its peripheral region and, thus, provides a relatively smaller marker image. Since, with the marker array described herein, the non-reflective object (e.g., a sphere) does not have to be coated with a retro-reflective film, it also can be used for other purposes, such as a marker for determining a position by means of a laser tracker (high-precision laser range tracker) or a mechanical coordinate measuring apparatus, for example.

Since it is possible to use a plane or flat reflective surface, the production costs for a marker can be reduced.

The reflective surface is preferably retro-reflective and exhibits, for example, a circular or rectangular (e.g., a square) surface, wherein the center point of the circle or in the barycenter of the reflective surface, a fixing element protruding, for example, perpendicularly from the reflective surface can be arranged, to which the absorbing object may be attached.

Advantageously, at least two and for example three or more markers as described herein can be used, which can serve as a reference star, for example. Two, three or more non-reflective, low-reflective or light-absorbing objects having the same or different geometries can then be arranged over a single or a number of different reflective surfaces, at the same distance or at different distances to the reflective surface.

In accordance with another aspect of the invention, there is provided an optical, for example, passive navigation system, comprising preferably at least one light source for emitting light, wherein any form of electromagnetic waves may be understood as light, such as for example light in the visible or infrared range. The navigation system also comprises at least one marker array as described herein, wherein the light emitted by the light source and reflected by the reflective surface can be detected by at least one camera. The camera can be connected to a computational unit which evaluates the optical signals detected by the camera and recognizes the dark or non-reflective region within the reflective surface generated by the absorbing object lying above the reflective surface in the range of vision of the camera. The computational unit then, based on known information on the marker, marker array, or reference star, can calculate the spatial position of the marker, marker array or of a reference star. The known information can include, for example, the geometry of the marker, marker array or reference star (e.g., the known information, stored in a database, on the shape of the absorbing object and its distance from the reflective surface).

In accordance with another aspect of the invention, there is provided a method for detecting a marker array as described herein, which ascertains whether or not an image or partial image of a reflective surface, detected by a camera, comprises an absorbing object at least partially shielding the surface, and verifies whether the but low-reflective or non-reflective region is completely surrounded by a reflective region. If the non-reflective region is completely surrounded by a reflective region, then the position of the absorbing object can be determined as the absorbing object, for example, by determining the center point of a circle (when using a sphere as the non-reflective object). If the non-reflective region is not completely surrounded by a reflective region, then it may be recognized that, viewed from the angle of view of the detection camera, the absorbing object is no longer completely situated in front of a reflective surface, such that the detection data of the camera may be recognized as defective. If it is thus established that the mapping of the absorbing object is drifting from the reflective surface, then an error can be detected, which can exclude the risk of incorrect detection, e.g., incorrectly determining the position of a marker.

The method is preferably used to calibrate, for example, a camera, a navigation system, a medical instrument or a patient to which the markers are connected. Marker movement relative to a calibration device (e.g., an infrared camera) is acceptable provided that the movement maintains the non-reflective region of the marker within the reflective region. In other words, the movement of the markers (or the calibration device) does not create a viewing angle that moves the perceived image of the non-reflective object outside the perceived image of the reflective region.

In accordance with another aspect of the invention, there is provided a computer program which, when loaded onto a computer or running on a computer, performs the method described herein. The invention also provides a program storage medium or computer program product comprising such a program.

The invention is described below on the basis of an example embodiment.

### BRIEF DESCRIPTION OF THE DRAWINGS

The forgoing and other embodiments of the invention are hereinafter discussed with reference to the drawings.

FIG. 1 illustrates an exemplary marker in accordance with the invention, and an exemplary navigation system for detecting a position of the marker.

### DETAILED DESCRIPTION

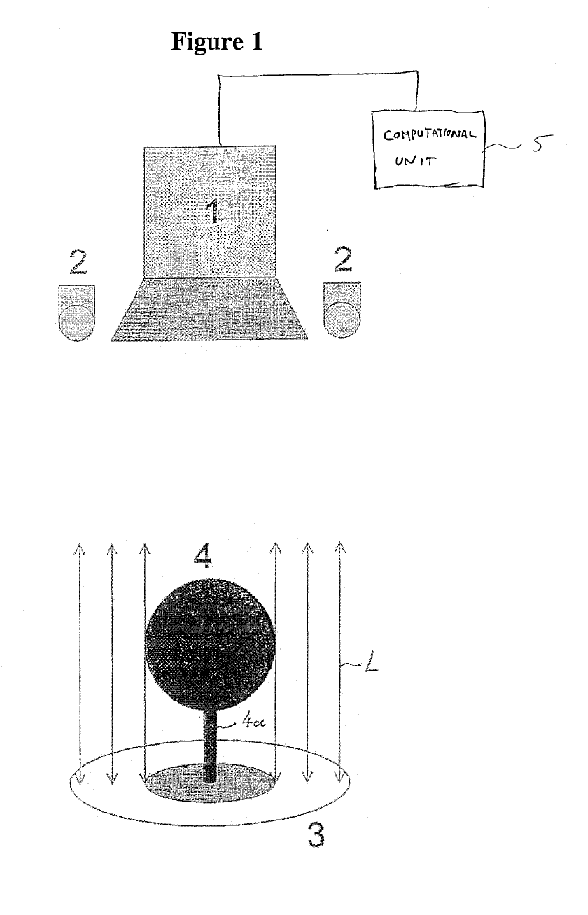

FIG. 1 shows a circular retro-reflective area 3 to which a spacer element 4a protruding perpendicularly from the reflective area 3 is fixed at a center point of the retro-reflective area, wherein a light-absorbing sphere 4 serving as an absorbing object is arranged on the spacer element 4a. Light L is emitted by light emitters 2 onto the marker array 3, 4 shown and is only reflected by the reflective area 3 in the region not covered by the light-absorbing sphere 4. This reflection pattern can be detected by a video camera 1 which, on the basis of the reflection image (which in the example embodiment shown is annular), can recognize the spatial position of the marker 3, 4 or of a reference star formed from a number of markers. The video camera 1 can provide the detected image data to a computational unit 5, which proceeds to calculate a position of the marker array 3, 4 in three dimensional space.

Thus, in addition to a reflection image, a reflection shadow generated by one or more absorbing objects 4 can be detected. This enables a sharp delineation between the reflective and the low-reflective or non-reflective region of the reflective area 3, which can increase the accuracy of detection.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A marker for a navigation system, comprising:
a reflective surface; and
a light-absorbing, non-reflective or low-reflective object arranged at a distance from the reflective surface.

2. The marker according to claim 1, wherein the distance between the object and the surface is variable.

3. The marker according to claim 1, wherein the object is arranged between the reflective surface and a light source, whereby the object creates a shadow on the reflective surface when light is emitted the light source onto the marker.

4. The marker according to claim 1, wherein the reflective surface is a plane or curved surface.

5. The marker according to claim 1, wherein the absorbing object is a sphere or has a spherical shape.

6. The marker according to claim 1, wherein the absorbing object is fixedly connected to the reflective surface by a connecting element.

7. The marker according to claim 1, wherein the reflective surface is circular, rectangular or square in shape.

8. The marker according to claim 1, wherein the reflective surface is retro-reflective.

9. A marker array comprising at least two or three markers according to claim 1.

10. A navigation system, comprising:
at least one marker according to claim 1;
at least one camera operable to detect a reflection image of the at least one marker; and
a computational unit coupled to the camera and operable to determine a spatial position of the marker based on a reflection image of the at least one marker.

11. The navigation system according to claim 10, further comprising at least one light source for illuminating the at least one marker.

12. A method for detecting a position of a marker or marker array that includes a reflective surface and a light-absorbing, non-reflective or low-reflective object arranged at a distance from the reflective surface, comprising:
ascertaining whether an area of the marker which does not reflect light is completely surrounded by a light-reflective area in the reflection image; and
concluding data obtained from the marker is defective if the area of the marker that does not reflect light is not completely surrounded by the light-reflective area.

13. The method according to claim 12, further comprising using the method to calibrate a camera, a navigation system, an instrument or a body.

14. A program embodied on a computer readable medium for detecting a position of a marker or marker array that includes a reflective surface and a light-absorbing, non-reflective or low-reflective object arranged at a distance from the reflective surface, comprising:
code that ascertains whether an area of the marker which does not reflect light is completely surrounded by a light-reflective area in the reflection image; and
code that concludes data obtained from the marker is defective if the area of the marker that does not reflect light is not completely surrounded by the light-reflective area.
